(19) United States
(12) Patent Application Publication
(10) Pub. No.: US 2005/0263651 A1
(43) Pub. Date: Dec. 1, 2005

(54) Captive retainer and method of using this retainer

(75) Inventor: Gilles Duponchelle, Savigny Sur Orge (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: SNECMA MOTEURS, Paris (FR)

(21) Appl. No.: 11/136,373

(22) Filed: May 25, 2005

(30) Foreign Application Priority Data
May 26, 2004 (FR) 04 51036

(51) Int. Cl. A61B 17/06; F16L 3/22

(52) U.S. Cl. 248/68.1; 248/65

(57) ABSTRACT

The retainer for fixing a long element (8) onto a surface comprises a retaining part (24, 28) which contains one long element (8), and at least one attachment flat (26). The retaining part (24, 28) is closed. It is preferably made from a material such that it can be attached by welding, and the attachment flats (26) comprise at least one lip (30).

### TECHNICAL FIELD

The invention relates to a retainer that comprises a retaining part that will hold at least one long element, and at least one attachment flat for fixing this retainer onto a surface.

Retainers are frequently used for attachment of long elements, for example electrical conducting wires, onto a surface. These retainers have one or two attachment flats that are used to fix them onto the surface by an appropriate means and a retaining part that holds one or several long elements in contact with the surface.

However, retainers known at the present time have a number of disadvantages. It is inconvenient to put them into place because the retainer has to be held with one hand while the other hand is used to fix it onto the surface, which is a difficult operation, particularly if the available space is small or if the surface onto which the retainer is to be fixed is inclined or vertical.

If the retainer drops, it has to be picked up which can cause additional operations to remove some parts which can cause a loss of time. This is why it is sometimes necessary to design or make a special tool, for example a cradle or a support used to hold the surface on which the retainers are to be fixed horizontal to make it easier to put them into place. However, the design of such a support requires additional time and costs.

The purpose of this invention is a retainer that overcomes the disadvantages of prior art.

These purposes are achieved according to the invention by the fact that the retainer that includes a closed retaining part to hold at least one long element and at least one attachment flat to fasten this retainer onto a surface, comprises a main part comprising at least one protrusion and at least one ring mounted in the protrusion.

The section of the retaining part may have various shapes but it is preferably approximately circular.

Due to these characteristics, the retainers are inserted on the long element, for example the electrical conductor. Considering that the retainer holding part is closed, these retainers are held in place by the electrical conductor and cannot escape. Consequently, if the retainer escapes during a placement operation, it is easy and fast to recover it because it cannot drop into structures located lower than the surface onto which the retainers have to be fixed. The result is a time saving and an improvement in the fastening quality of the long elements, for example electrical conductors.

Furthermore, it is not necessary to study, design and manufacture a special support tool for making the surface onto which the retainers will be fixed horizontal, which results in an additional saving.

Advantageously, the dimensions of the retaining part are chosen such that the long element(s) are held in place with a clearance.

Due to this characteristic, the long elements, for example electrical conductors, are held in place without being squeezed. Therefore they can easily slide inside the retainers, for example if they have to be removed. It is also possible to arrange several long elements in the same retainer.

Advantageously, the retainer is made from a material such that it can be attached by welding and the attachment flat(s) comprises at least one lip.

The ring may be longer than the width of the retaining part.

The method of fixing the long elements, particularly electrical conductors, onto a metallic surface is characterised in that:
a stop element is placed on the long element;
an appropriate number of retainers according to the invention are inserted onto the long element;
the long element is placed on the surface, with the stop element being placed near the bottom;
a first retainer located in the lowest position is lifted to an attachment position using an electrode bearing on a lip of the retainer;
the retainer is welded onto its attachment position;
the above two operations are repeated for the retainer immediately above the first retainer and so on until all retainers have been fixed.

Other characteristics and advantages of the invention will become clear after reading the following description of example embodiments given for illustrative purposes with reference to the attached Figures. In these Figures:

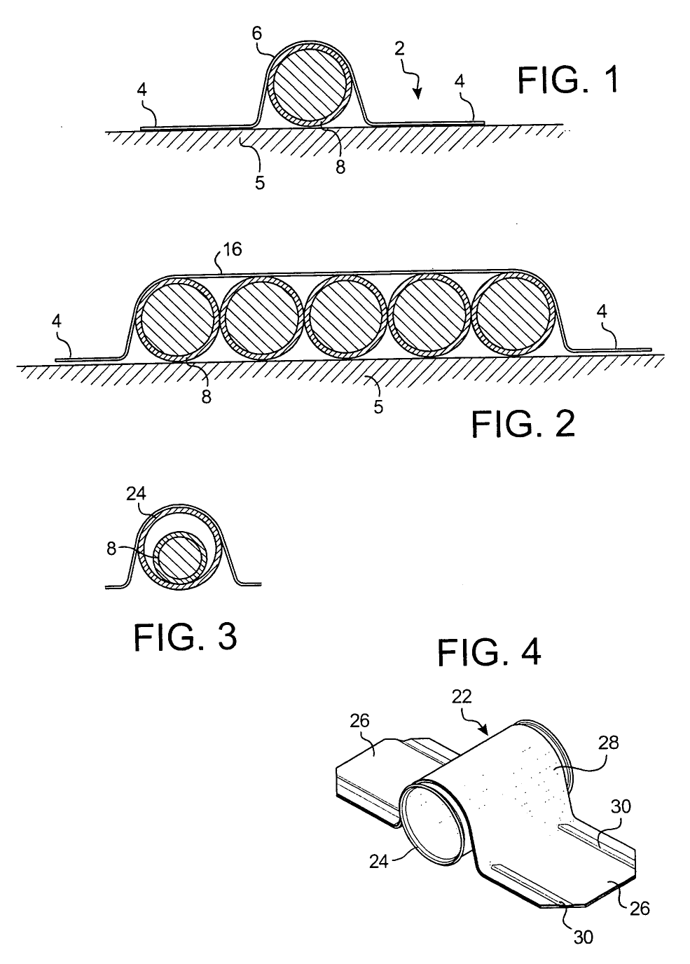

FIGS. 1 and 2 show retainers according to prior art;

FIG. 3 is an end view of a retainer according to this invention;

FIG. 4 is a perspective view of a variant embodiment of the retainer in FIG. 3;

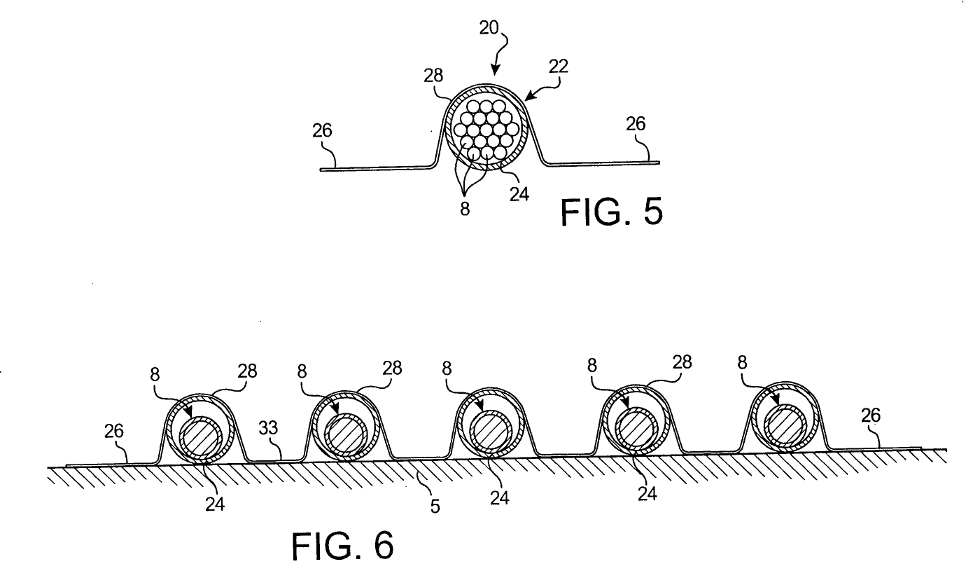

FIG. 5 shows a variant in which several conductors will be placed;

FIG. 6 is another embodiment in which several conductors will be placed;

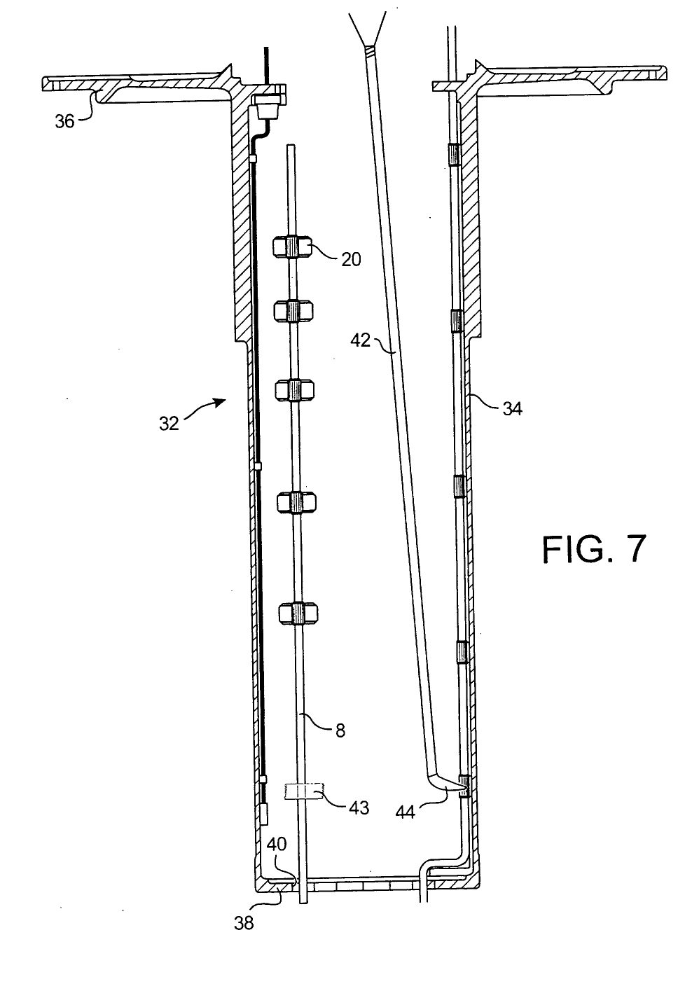

FIG. 7 shows a sectional view of an instrumentation rod on which retainers according to the invention have been fixed.

FIG. 1 shows an example of a retainer according to prior art. It includes two flats 4 used to fix it onto a surface, for example using nails or screws passing through the two flats 4, or by spot welds onto a metallic surface 5. A retaining part 6 in the form of an inverted V rounded at its end holds the long element 8, for example an electrical conductor, in position. A retainer of this type can drop while it is being placed, in particular if it is fixed on an inclined or vertical surface.

FIG. 2 shows another known embodiment of a retainer according to prior art. In this embodiment, the retaining part 16 has an elongated shape so that several electrical conductors 8, in the example shown there are five, can be placed inside it side by side.

However, the attachment of this type of retainer is even more difficult than the retainer shown in FIG. 1 because the electrical conductors 8 have to be held in place parallel to each other during the attachment operation.

FIG. 3 shows a first example embodiment of a captive retainer according to the invention. It is composed of two parts, namely a main part 22 and a retaining part 24, in this example a ring. As a variant, the retainer could be made from a single part, bearing in mind that the retaining part must be closed.

The main part 22 comprises two flats 26 used to fasten the retainer onto a surface and a protrusion 28 arranged between the two flats 26. The ring 24 is placed inside the protrusion 28. It is assembled to the main part, for example by spot welding. It may be in various shapes but it is preferably circular. In this embodiment, the inside diameter of the ring is chosen to enable it to contain a single conductor. Initially, the retainers are slid onto the electrical conductor 8. Considering that the conductor 8 is contained in the ring 24, it is possible to provide a clearance so as to enable easy sliding of the retainer. In a second step, the electrical conductor 8 onto which the retainers 20 were slid is placed on the surface and the attachment flats 26 are fixed onto this surface.

FIG. 4 shows a perspective view of a variant embodiment of the retainer in FIG. 3. In this variant, the flats 26 comprise lips 30 at each of their ends. Furthermore, the length of the ring 24 is greater than the width of the main part 22 such that the ring 24 projects beyond the main part on each side of it. The variant embodiment in FIG. 4 is particularly suitable for welding retainers onto a metallic surface. They can be displaced using an electrode bearing on a lip 30 until they reach the position at which they are to be fixed and then welded in this position. A retainer of this type may be fixed in locations that are difficult to access because there is no need to insert a hand to fix it, all that is necessary is to pass the electrode.

FIG. 5 shows a variant embodiment of the retainer in FIG. 3 or 4. This variant is distinguished by the fact that the inside diameter of the ring 24 is sized to hold several conductors, for example eight or even more, instead of only one. Furthermore as described above, since the conductors are contained in the ring, a clearance can be left between the conductors and the ring. Thus, the conductors can slide. It is easy to place them because their placement is made in two operations. In the first step, the complete set of retainers is slid onto the conductors. In the second step, the conductors onto which the retainers have been slid are placed on the surface and the attachment flats 26 are welded.

FIG. 6 shows another embodiment of the retainer according to the invention that will contain several conductors. It does not include a single ring for all wires 8, but rather one ring for each wire. A minimum number of spot welds is required to satisfactorily attach each ring 24 on the protrusions 28, and in the example there are three rows of three spot welds. Furthermore, the retainer is fastened to the surface by spot welding onto the flats 26, but also onto the plane connection parts 33 located between two consecutive protrusions 28. As described before, due to the fact that each conductor is contained in a ring, a clearance can be left so that they can slide. As described above, placement is done in two operations. Firstly, each conductor is slid into the ring corresponding to it, and the conductors on which the retainers were slid are then placed on the surface and the attachment flats 26 and the intermediate parts 33 are welded.

FIG. 7 illustrates application of the retainers according to the invention to routing of measurement loops, simply called “loops” in the trade, in an instrumentation rod that may for example be used for measuring operating parameters of an aircraft engine. The instrumentation rod, denoted as a whole by the general reference 32, comprises an elongated cylinder 34 arranged vertically and an attachment flat 36 located at an open end of the cylinder 34. The bottom 38 of the cylinder 34 comprises one or several holes 40 for loops 8 to pass through. The loops 8 will be connected to parts of the engine (not shown) to which the rod is related. The loops 8 are held on the inner surface of the steel cylinder 34 by a series of electrically welded retainers 20. The diameter of the cylinder 34 is too small for a hand to be inserted inside it, consequently the retainers are moved using an electrode 42 on an electric welding station. This is done by using a stop element 43, for example a piece of adhesive tape, on the bundle of loops 8 and then the retainers 20 are slid one after the other onto the bundle of loops like beads. The assembly is inserted inside the rod, with the stop element 43 being placed near the bottom 38. The next step is to take the retainer located immediately above the stop element using the tip 44 of the electrode 42 and to lift it up bearing on one of the lips 30 provided on the flats 26 (FIG. 4), up to the position at which it is to be fixed, and it is then welded in this position. The same operation is then repeated with the retainer located immediately above the first retainer and so on until all retainers have been fixed at a uniform spacing from each other.

The retainer that has just been described can create a time saving and improve the routing quality of loops 8. Since part of the work is done before routing inside the rod, the global instrumentation time is shortened. The diameter of the retainers varies as a function of the number of loops 8 and the diameters used for routing. Loops 8 can also be slid during disassembly leaving the retainers in place, which prevents them from being damaged.

1. Retainer for fixing at least one long element (8) onto a surface, comprising at least a closed retaining part (24, 28) which contains at least one long element (8), and at least one attachment flat (26), characterised in that it comprises a main part (22) comprising at least one protrusion (28) and at least one ring (24) mounted in the protrusion (28).

2. Retainer according to claim 1, characterised in that the retaining part (24, 28) has a circular cross-section.

3. Retainer according to claim 1 or 2, characterised in that the dimensions of the retaining part (24, 28) are chosen such that the long element(s) (8) is (are) held in place with a clearance.

4. Retainer according to one of claims 1 to 3, characterised in that it comprises a plurality of retaining parts (24, 28) separated by connection parts (33).

5. Retainer according to one of claims 1 to 4, characterised in that it comprises two flats (26) located on each side of the retaining part(s).

6. Retainer according to one of claims 1 to 5, characterised in that it is made from a material with that it can be attached by welding.

7. Retainer according to one of claims 1 to 6, characterised in that the attachment flat(s) (26) comprise(s) at least one lip (30).

8. Retainer according to one of claims 1 to 7, characterised in that the ring (24) is longer than the width of the retaining part (28).

9. Method of fixing at least one long element (8), particularly electrical conductors, onto a metallic surface, characterised in that:
a stop element (43) is placed on the long element (8);
an appropriate number of retainers (20) according to claim 8 or 9 is inserted onto the long element;
the long element is placed on the surface, with the stop element (43) being placed near the bottom;
a first retainer located in the lowest position is lifted to an attachment position using an electrode (42) bearing on a lip (30) of the retainer;
the retainer is welded onto its attachment position;
the above two operations are repeated for the retainer immediately above the first retainer and so on until all retainers have been fixed.
